(19) United States
(12) Plant Patent Application Publication
(10) Pub. No.: US 2009/0126055 P1
(43) Pub. Date: May 14, 2009

(54) PENSTEMON PLANT NAMED 'PENHARED'

(50) ;

(75) Inventor: Martien Everett Gutter, Enkhuizen (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/982,757

(22) Filed: Nov. 5, 2007

(51) Int. Cl. A01H 5/00

(52) U.S. Cl. PLT/465

(57) ABSTRACT

A new Penstemon plant, particularly distinguished by its red colored flower with white throat, early flowering, and good branching habit.

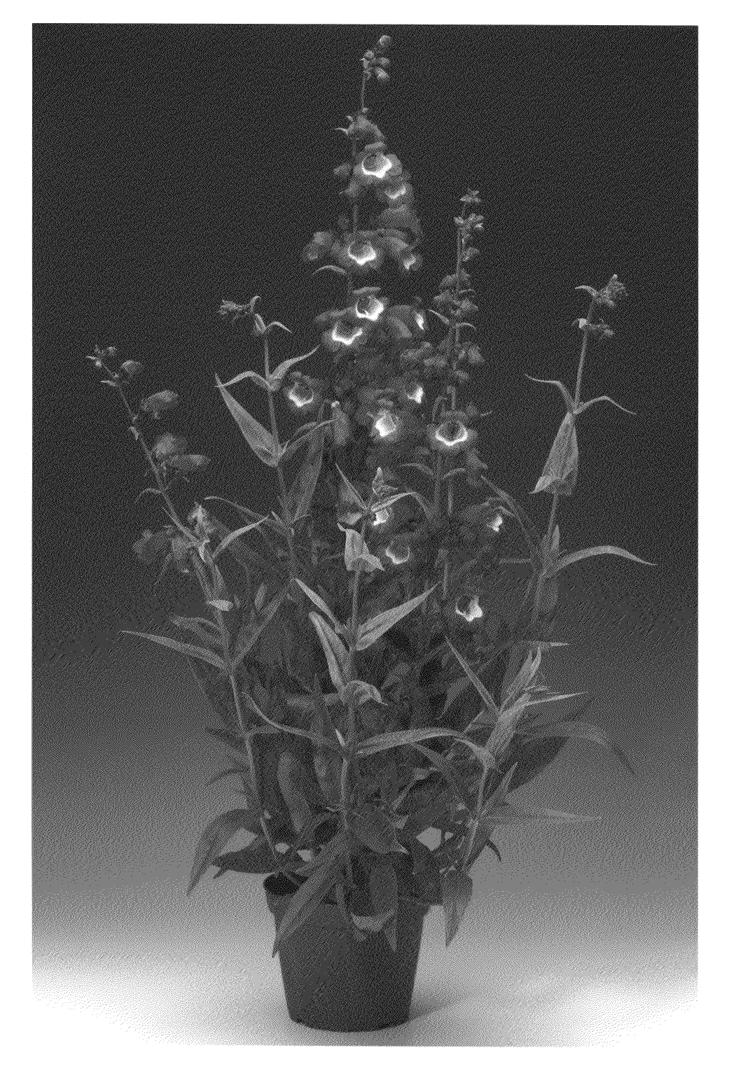

### LATIN NAME OF THE GENUS AND SPECIES OF THE PLANT CLAIMED

Penstemon hartwegii

### VARIETAL DENOMINATION

‘Penhared’

### BACKGROUND OF THE NEW PLANT

The present invention comprises a new distinct cultivar of Penstemon, botanically known as Penstemon hartwegii.

The new Penstemon is a product of a planned breeding program conducted in Enkhuizen, Netherlands. The new Penstemon cultivar has a well branched plant habit, has a red flower with white throat, and is early flowering.

The new Penstemon is propagated by cuttings resulting from a single seedling. This seedling originates from a selection in Penstemon hartwegii ‘Picotee’ (not patented). This selection was made through one cycle of half sib selection in the period June 2002 until July 2003 in Enkhuizen, Netherlands. The seedling was selected in July 2003 in Enkhuizen, Netherlands.

The present cultivar has been repeatedly asexually reproduced by cuttings in Enkhuizen, Netherlands, since 2003. The distinctive characteristics of this new Penstemon are stable and reproduced true to type in successive generations of asexual reproduction.

The new Penstemon plant is a perennial in zones 4 to 9 in the US.

### DESCRIPTION OF THE DRAWING

The new Penstemon plant is illustrated by the accompanying photographic drawing which shows blooms, buds and foliage of the plant in full color, the color showing being as true as can be reasonably obtained by conventional photographic procedures.

### BOTANICAL DESCRIPTION OF THE PLANT

The following detailed descriptions set forth the distinctive characteristics of this new Penstemon. The data which defines these characteristics were collected from asexual reproductions carried out in Enkhuizen, Netherlands. The plant history was taken on 15 week old plants. The instant plant was grown in an 11 cm container in an outdoor container field.

Color references are primarily to the R.H.S. Colour Chart of The Royal Horticultural Society of London.

Plants of the new Penstemon differ primarily from the plants of the originator ‘Picotee’ in the following characteristics:

Plants of the new Penstemon start flowering earlier and are more branched than plants of the originator ‘Picotee.’

TABLE 1
DIFFERENCES BETWEEN THE NEW CULTIVAR
‘PENHARED’ AND A SIMILAR CULTIVAR

| | ‘Penhared’ | ‘Rubycunda’ (Not patented) |
|---|---|---|
| Earliness | Early | Late |
| Pubescence of throat | Long hairs | Short hairs |
| Branching | Very good | Medium |

The plant:
   Classification — botanical.—Penstemon hartwegii.
Parentage:
   Originator selection.—‘Picotee’ (Not patented).
Propagation:
   Type of cutting.—Terminal cuttings.
   Time to initiate roots.—12-16 days at 18° C.
   Time to produce a rooted cutting.—3 weeks.
   Root description.—Fine, fibrous and white in color.
Plant description:
   Form.—Compact basal rosette.
   Hardiness.—USDA Zone 4 to 9.
   Size.—48 cm tall and 26 cm wide.
   Habit.—Suffrutescent.
The foliage:
   Type.—Simple.
   Arrangement.—Opposite.
   Shape.—Lanceolate.
   Venation.—Pinnate.
   Margins.—Entire, undulate.
   Apex.—Acuminate.
   Base.—Sessile, obtuse.
   Length.—9.1 cm.
   Width.—2.9 cm.
   Vestiture.—Glabrous.
   Texture.—Leathery.
   Leaf color.—Upper surface: 137A Lower surface: 143B.
The inflorescence:
   Type.—Thryse and virgate.
   Number of flowers.—6-43 per cyme.
   Peduncle.—Height: 43 cm Diameter: 3 mm Color: 144B Texture: Glabrous.
   Pedicel.—Length: 1-1.5 cm Diameter: 1-2 mm Texture: Glabrous Color: 146C.
   Flowering period.—From June until August.
   Flower bud.—Length: 3.7 cm Width: 3.8 cm Position: Closed tubular Color: 45C, center N155A.
   Flower type.—Zygomorphic.
   Flower shape.—Tubular, bilabiate.
   Flower tube.—Length: 0.5 cm Width: 0.2 cm.
   Number of flower lobes.—Five, 2 upper lobes, three lower lobes.
   Flower size.—Length: 3.7 cm Width: 3.8 cm.
   Flower color.—45C, center N155A.
   Calyx.—Number of lobes: 5 overlapping lobes Length: 0.7 cm Width: 0.5 cm Color: 137A.
   Calyx lobes.—Apex: Acuminate Margin: Entire.
   Lastingness.—On the plant: Each cyme blooms for about 4 weeks on the plant In the vase: 3 days.
Reproductive organs:
   Stamen.—Number: 5 Shape: Dehiscent.
   Anther color.—158A.
   Pollen color.—158D.
   Pistil length.—2.7 cm.
   Pistil color.—64B.
   Fragrance.—None.
   Filament length.—2.5 cm.
   Filament color.—155B.
Seed development: Seed development has not been observed to date.
Disease tolerance: No disease/pest resistance has been observed to date.
Weather resistance: The new Penstemon plant is resistant to temperatures ranging from −6° C. to +40° C.

What is claimed is:

1. A new and distinct cultivar of Penstemon plant named ‘Penhared,’ as substantially illustrated and described herein.
